(19) United States
(12) Patent Application Publication
(10) Pub. No.: US 2023/0243848 A1
(43) Pub. Date: Aug. 3, 2023

(54) Biomarkers Predictive of Atopic Dermatitis

(71) Applicant: Johnson & Johnson Consumer Inc., Skillman, NJ (US)

(72) Inventors: Russell Gould, Yardley, PA (US); Georgios N. Stamatas, Issy-les-Moulineaux (FR); Janeta Nikolovski, Princeton, NJ (US); Pierre-Francios Roux, Juvignac (FR); Imane Lboukili, Issy-les-Moulineaux (FR); Gabriel Al-Ghalith, Medford, MA (US); Takahiro Sato, Philadelphia, PA (US)

(21) Appl. No.: 18/100,061

(22) Filed: Jan. 23, 2023

Related U.S. Application Data
(60) Provisional application No. 63/304,290, filed on Jan. 28, 2022.

(51) Int. Cl. G01N 33/68

(52) U.S. Cl. CPC G01N 33/6881; G01N 2800/202; G01N 2800/52; G01N 2800/50

(57) ABSTRACT

A method to predict a propensity of an individual to develop atopic dermatitis is disclosed. The method, which involves use of biological markers, can also be used to evaluate the efficacy of a composition to treat atopic dermatitis.

### CROSS RELATED APPLICATION

The present application claims the benefit of the earlier filing date of U.S. provisional patent application 63/304290, filed Jan. 28, 2022, the entirety of which application is hereby incorporated by reference herein as if fully set forth herein.

### FIELD OF THE INVENTION

The invention relates to factors that can be used to predict the propensity of an individual to develop atopic dermatitis. The invention also relates to methods of using the factors to evaluate the potential of a skin treatment regimen, ingredient and/or composition to prevent atopic dermatitis.

### BACKGROUND

The skin is a set of cells and macromolecules grouped together in the form of a resistant and flexible tissue which covers the entire body. It is made up of two joined layers, the epidermis and the dermis, and associated subcutaneous tissues.

The main function of the skin is to establish a protective barrier against environmental insults while allowing some exchanges between the internal and external environment. The barrier function is particularly important in limiting epidermal water loss. This function is provided chiefly by the corneal layer (stratum corneum), the uppermost layer of the epidermis, composed of flattened, anucleate cells called corneocytes. The watertightness of this “brick wall” is provided by an intercellular cement composed of specific lipids (cholesterol, cholesterol sulphate, free fatty acids and ceramides). The regenerative capacity of the epidermis is conferred by adult stem cells which allow regular replacement of the differentiated cells eliminated during keratinization. This process is particularly crucial for barrier function maturation and maintenance.

Adaptation to extrauterine life is a process which begins at birth and continues throughout the first year of life. The first months of postnatal life are a period of structural and functional reorganization of the skin allowing physiological adaptation to the extrauterine environment. For example, the immaturity of newborn skin is highlighted by the difference in the structure and molecular composition of the stratum corneum compared with that of adults. These are incomplete and thus continue to develop for at least the first 12 months after birth (Chiou et al., Skin Pharmacol Physiol, 17: 57-66, 2004; Nikolovski et al., J Invest Dermatol, 128: 1728-1736, 2008; Stamatas et al., Pediatr Dermatol, 27: 125-131, 2010; Telofski et al., Dermatol Res Pract, 2012: 198789, 2012). In addition, the results of two recent clinical studies (Fluhr et al., Br J Dermatol, 166(3): 483-90, 2012 and Fluhr et al., Br J Dermatol, 2014, 171(5): 978-86) suggest that infant skin presents a certain immaturity in its ability to capture water and regulate related mechanisms. Moreover, these studies have shown that the epidermal barrier organizes structurally from birth to 2 years of age and is therefore not completely mature during this period. This helps to explain the fragility of infants' and young children's skin and its susceptibility to chemical, physical and microbial attacks.

In addition, incomplete skin maturation can have significant clinical consequences. It is therefore important to allow the skin to be constructed and to develop properly and harmoniously, otherwise its functional and structural organization could be compromised. In this respect, it is crucial to preserve the barrier function and the renewal capacity of the epidermis.

Thus, the immaturity of the barrier and of the mechanisms regulating hydration in a baby's skin contributes to make it even more vulnerable to pathological situations such as atopic dermatitis.

Atopic dermatitis is one of the most common chronic diseases in the population. It is characterized by a set of clinical signs, the most important of which are pruritus and eczematous lesions, which may be acute, subacute or chronic. It almost always begins in infants or young children, while the barrier is structurally and functionally organizing itself. Atopic dermatitis usually begins at around three months of age, but sometimes in the first few weeks of life. It progresses in alternating relapse and remission phases. Depending on the child and the severity of the condition, it may last from several months to several years. A small percentage may persist into adulthood.

Atopic dermatitis is, first and foremost, a chronic inflammatory dermatological disease combining impairment of the skin barrier and skin inflammation. In a first sensitization phase, the skin barrier defect allows allergens to penetrate through the skin. Allergens that penetrate the upper layers of the epidermis are processed (internalized) by epidermal Langerhans cells and dermal dendritic cells. Langerhans cells are antigen-presenting cells that are able to capture skin antigens, prepare them and present them to T lymphocytes. This presentation leads to activation of the Th2 response, which results in the production of inflammatory cytokines such as IL-4, IL-5 and IL-13 (see for example Bieber, Ann Dermatol. 2010, 22(2): 125-137).

Once the individual has been sensitized cutaneously, subsequent contact with the allergen in question may induce eczema lesions. This response is also mediated by the Th2 response. In particular, Langerhans cells present the peptides to specific T lymphocytes that, when activated, produce Th2 cytokines (IL-4, IL-5). The resulting cytokines will recruit new cells, including eosinophils, which play an important role in the development and chronicity of eczema lesions.

In all periods of activity of the disease, bacterial or viral skin superinfections are the most common complications. The skin of atopic dermatitis patients is highly susceptible to secondary infections, which then tend to become more widespread. For example, the bacterium Staphylococcus aureus is a major cause of skin infections. It commonly colonizes the skin of atopic dermatitis patients, whereas it is only transiently present on healthy skin. The bacterium then secretes virulence factors that further reduce the barrier function, exacerbating the disease and contributing to its chronicity. In addition, S. aureus is usually found in atopic dermatitis patients in the form of homogeneous biofilms, a form resistant to host defenses and treatments.

U.S. Pat. No. 10,175,230 to Laboratoires Expanscience discloses a method for assessing the effectiveness of a C7 sugar or derivative thereof in the prevention and/or treatment of at least one deficiency of the skin barrier of a subject.

U.S. Published Application No. 20190242880 to Laboratoires Expanscience discloses methods for evaluating the in vitro efficacy of formulations in preventing the effects of dehydration on children's skin.

French Published Application No. 2792728 to L'Oreal discloses a method of evaluating the effects of a product on epidermal lipogenesis that includes applying the product to the surface of a skin equivalent, measuring the variation of a marker of epidermal lipids, then making a comparison with a similar measurement for a control sample.

United States Patent Application No. 20020182112 to Unilever Home & Personal Care USA discloses an in vivo method for measuring the binding of chemical compounds or mixtures of compounds to skin constituents.

U.S. Pat. No. 8,053,003 to Laboratoires Expanscience discloses a method of treating sensitive skin, irritated skin, reactive skin, atopic skin, pruritus, ichtyosis, acne, xerosis, atopic dermatitis, cutaneous desquamation, skin subjected to actinic radiation, or skin subjected to ultraviolet radiation, comprising administering an effective amount of a composition comprising furan lipids of plant oil and thereby increasing synthesis of skin lipids.

U.S. Pat. Nos. 9,808,408 and 10,172,771 to The Procter & Gamble Company discloses a method of identifying a rinse off personal care composition that includes: (a) generating one or more control skin profiles for two or more subjects; (b) contacting at least a portion of skin of the subjects with a rinse-off test composition, rinsing the test composition off the portion of skin, extracting one or more skin samples from each of the subjects, and generating from the extracted samples one or more test profiles for the subjects; (c) comparing the one or more test profiles to the one or more control profiles and identifying the rinse-off test composition as effective for improving the stratum corneum barrier in a human subject who shows (i) a decrease in one or more inflammatory cytokines, (ii) an increase in one or more natural moisturizing factors, (iii) an increase in one or more lipids, and (iv) a decrease in total protein.

Ring J. (2016) Pathophysiology of Atopic Dermatitis/Eczema. In: Atopic Dermatitis. Springer, Cham PMID:16098026, discloses the state of the art in research in atopic dermatitis, or atopic eczema.

Glatz et al., Emollient use alters skin barrier and microbes in infants at risk for developing atopic dermatitis, PLoS ONE, 13(2):e0192443 (2018), discloses that emollient use correlated with an increased richness and a trend toward higher bacterial diversity as compared to no emollient use in infants at risk for developing atopic dermatitis.

Capone et al., Effects of emollient use on the developing skin microbiome, presented at the American Academy of Dermatology Annual Meeting, 1-5 Mar. 2019, Washington DC, USA, discloses that microbial richness is significantly greater with infant wash and lotion than with wash alone. Capone et al. also discloses that both cleansing alone and cleansing and emollient regimens were well tolerated; skin pH remained slightly acidic throughout the study in each regimen; no significant changes for dryness, redness/erythema, rash/irritation, tactile roughness or total score of objective irritation or for overall skin appearance, in either group vs. baseline at any timepoint; an increase in microbial richness seen by 2 and 4 weeks with wash and by 4 weeks with addition of lotion; by 4 weeks use, lotion use increased richness more than wash alone; mild infant wash+lotion routine may best help improve microbial richness, which may contribute to overall skin barrier health by providing the right environment for healthy skin microbes to flourish.

R∅pke, Mads Almose, et al. “Non-invasive assessment of soluble skin surface biomarkers in atopic dermatitis patients—Effect of treatment.” Skin Research and Technology (2021), discloses the use of a non-invasive patch technique to collect chemokines and cytokines on the surface of lesional and non-lesional atopic dermatitis skin before and after topical treatment.

U.S. Pat. No. 10,226,499 to KAMEDIS LTD. and BIO-FD&C CO. LTD. discloses a method of treating atopic dermatitis that comprises administering a therapeutically effective amount of a composition comprising water extracts of Rheum palmatum, Cnidium Monnieri, Scutellaria baicalensis, Sanguisorbae officinalis, and Ailanthus altissima to upregulate expression of a human beta-defensin in a cell of the subject.

U.S. Published Application No. 20100016232 to Novozymes A/S discloses a method for treating an inflammatory disease such as atopic dermatitis that comprises administering a human beta-defensin, including human beta defensin 1.

U.S. Published Application No. 20110217249 to Dreher discloses a method for treating skin diseases and disorders associated with deregulation of the skin's antimicrobial peptide formation, processing, or both comprising administering an effective amount of one or more antimicrobial peptide sequestering compounds to a patient suffering therefrom, wherein the disease or disorder may be atopic dermatitis and wherein the antimicrobial peptide being sequestered by the compound may be a human defensin polypeptide.

U.S. Pat. No. 11,090,393 and U.S. Published Application No. 2019-0212324 to Johnson & Johnson Consumer Inc. disclose methods of evaluating the potential impact of a system on infant skin that includes use of a computational model of adult skin penetration to visualize penetration of a marker by optimizing penetration parameters so that the model of adult skin penetration profiles match the experimental data; and transferring the optimized penetration parameters to a computational model of infant skin.

U.S. Published Application No. 20200360259 to Johnson & Johnson Consumer Inc. discloses a method of screening a skin treatment regimen, ingredient and/or composition for benefit to skin that includes measuring the level of one or more small molecule metabolites in an area of skin prior to application of the skin treatment regimen, ingredient and/or composition.

U.S. Published Application No. 20200375888 to Johnson & Johnson Consumer Inc. discloses a method of evaluating an ability of a skin barrier system to protect infant skin from external irritants that includes use of a computational model of adult skin inflammation to visualize an effect of an external irritant by optimizing inflammation parameters so that the model of adult skin inflammation profiles match the experimental data; and transferring the optimized inflammation parameters to a computational model of infant skin. To date, there is no cure for atopic dermatitis. Treatments are primarily local, the aim of which is to improve symptoms and control disease progression (Eichenfield et al., J Am Acad Dermatol. 2014; 70(2): 338-351; Eichenfield et al., J Am Acad Dermatol. 2014; 71(1): 116-132). In particular, the daily use of emollients is essential to restore and protect the damaged skin barrier. Many different emollients are available on the market. However, the precise mechanisms by which they exert their beneficial effects are insufficiently understood. There thus remains a need to further understand the mechanisms and to select effective, well-tolerated emollients to not only treat, but hopefully also prevent atopic dermatitis.

### DESCRIPTION OF THE FIGURES

FIG. 1A to FIG. 1E are graphs showing expression levels of inflammatory cytokines measured from skin swabs from healthy high risk subjects that developed AD within 12 months (converters) and those that didn't (non-converters) (group B). FIG. 1A shows IL.36 g levels for both body and face for converters and non-converters. FIG. 1B shows IL.1RA levels for both body and face for converters and non-converters. FIG. 1C shows hBD1 levels for both body and face for converters and non-converters. FIG. 1D shows S100A8.9 levels for both body and face for converters and non-converters. FIG. 1E shows combined Z score cytokines for both body and face for converters and non-converters.

FIGS. 2A and 2B show odds ratios reflecting the development of AD with the combined Z score, Shannon Diversity (alpha diversity); TEWL; Corneometer; Birth Mode; Sex; and Age for body (FIG. 2A) and face (FIG. 2B), respectively.

FIGS. 3A and 3B are ROC curves for prediction of the development of AD using inflammatory cytokine data for body (FIG. 3A) and face (FIG. 3B), respectively.

FIGS. 4A and 4B shows trans epithelial water loss (TEWL) on face (FIG. 4A) and body (FIG. 4B), respectively.

FIGS. 5A and 5B show average total amino acids for group A, group B and group C, respectively.

Subjects that converted to AD display increased skin inflammation and microbial dysbiosis at baseline.

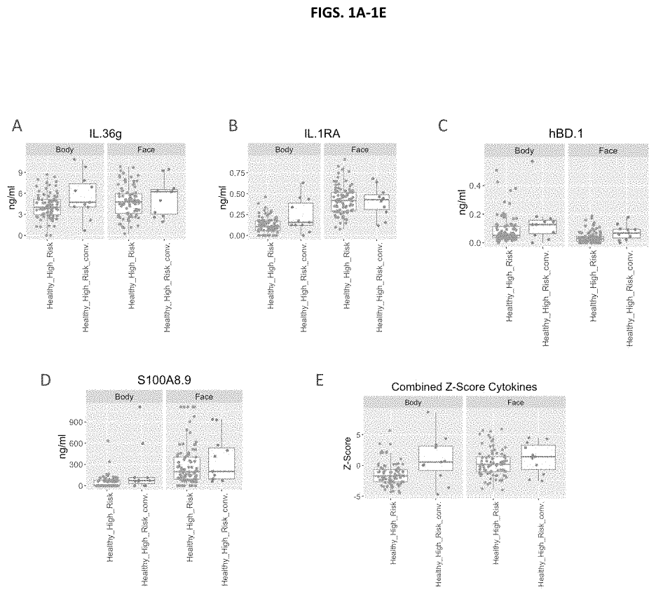

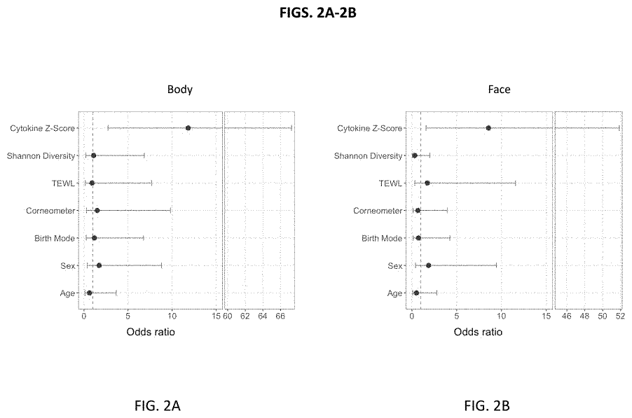

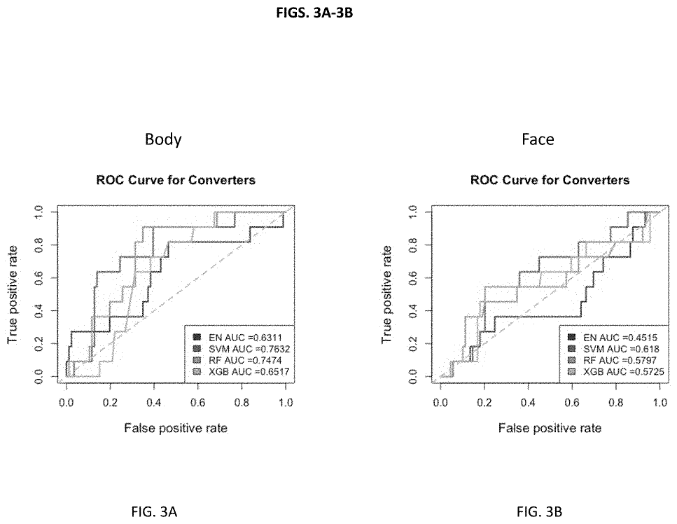

We identified four inflammatory cytokines (hBD1, IL1RA, IL36g, 5100A8/9) on the body and one on the face (hBD1) that had potential in distinguishing high-risk subjects at baseline that developed AD within 12 months vs. those that didn't develop AD (FIGS. 1A-D, p<0.3). A combined Z-score based on these 4 cytokines was significantly different between converters vs non-converters in body samples at baseline (FIG. 1E, p<0.05), and had an OR of 11.826 when dividing into a low and high cytokine Z-score group (FIGS. 2A and 2B). Machine learning models trained on predicting AD lesional subjects vs. healthy low-risk subjects using cytokine data and tested on healthy high-risk converters vs non-converters could predict development of AD on the body with AUCs ranging from 0.63 to 0.76 (FIGS. 3A and 3B). Samples collected from the face had a lower OR of 8.554 using the combined Z-Score (FIGS. 2A and 2B) and a lower predictive ability as evidenced by AUCs ranging from 0.45 to 0.58 (FIGS. 3A and 3B).

### DETAILED DESCRIPTION

### Definitions

As used herein, the following terms shall have the meaning specified thereafter:

“Alpha diversity” as used herein means diversity of species in different sites or habitats within a local scale.

“Biomarker” as used herein refers to any biological molecule (gene, protein, lipid, metabolite) that, singularly or collectively, reflect the current or predict future state of a biological system. Thus, as used herein, various biomarkers are indicators of the quality of skin. The ability to prevent and/or treat skin conditions can also be assessed by measuring one or more biomarkers.

“Consumer” as used herein refers to an individual who purchases and/or uses skin treatment regimens, ingredients and/or compositions in accordance with the disclosure. In some instances, therefore, a consumer may be alternately referred to herein as a “user.”

“Control” as used herein means a region of epithelial tissue which has not been contacted with and/or by a regimen, ingredient and/or composition which has contacted the affected surface.

“Effective amount” as used herein means an amount of a regimen, ingredient and/or composition sufficient to significantly induce a positive skin benefit, including independently or in combination with other benefits disclosed herein. This means that the content and/or concentration of active component in the regimen, ingredient and/or composition is sufficient that when the regimen, ingredient and/or composition is applied with normal frequency and in a normal amount, the regimen, ingredient and/or composition can result in the treatment of one or more undesired skin conditions. For instance, the amount can be an amount sufficient to inhibit or enhance some biochemical function occurring within the skin. This amount of active component may vary depending upon, among other factors, the type of regimen, ingredient and/or composition and the type of skin condition to be addressed.

“Emollient” as used herein refers to chemical agents specially designed to make the external layers of the skin (epidermis) softer and more pliable.

“Epidermis” as used herein refers to the outer layer of skin, and is divided into five strata, which include the: stratum corneum, stratum lucidum, stratum granulosum, stratum spinosum, and stratum basale. The stratum corneum contains many layers of dead, anucleated keratinocytes that are essentially filled with keratin. The outermost layers of the stratum corneum are constantly shed, even in healthy skin. The stratum lucidum contains two to three layers of anucleated cells. The stratum granulosum contains two to four layers of cells that are held together by desmosomes that contain keratohyaline granules. The stratum spinosum contains eight to ten layers of modestly active dividing cells that are also held together by desmosomes. The stratum basale contains a single layer of columnar cells that actively divide by mitosis and provide the cells that are destined to migrate through the upper epidermal layers to the stratum corneum. The predominant cell type of the epidermis is the keratinocyte. These cells are formed in the basal layer and exist through the epidermal strata to the granular layer at which they transform into the cells know as corneocytes or squames that form the stratum corneum. During this transformation process, the nucleus is digested, the cytoplasm disappears, the lipids are released into the intercellular space, keratin intermediate filaments aggregate to form microfibrils, and the cell membrane is replaced by a cell envelope made of cross-linked protein with lipids covalently attached to its surface. Keratins are the major structural proteins of the stratum corneum. Corneocytes regularly slough off (a process known as desquamation) to complete an overall process that takes about a month in healthy human skin. In stratum corneum that is desquamating at its normal rate, corneocytes persist in the stratum corneum for approximately 2 weeks before being shed into the environment.

“Epithelial tissue” as used herein refers to all or any portion of the epithelia, in particular the epidermis, and includes one or more portions of epithelia that may be obtained from a subject by a harvesting technique known in the art, including those described herein. By way of example and without being limiting, epithelial tissue refers to cellular fragments and debris, proteins, isolated cells from the epithelia including harvested and cultured cells.

“Filaggrin” (filament aggregating protein) as used herein refers to a filament-associated protein that binds to keratin fibers in epithelial cells. Filaggrin is essential for the regulation of epidermal homeostasis. Within the stratum corneum, filaggrin monomers can become incorporated into the lipid envelope, which is responsible for the skin barrier function. Alternatively, these proteins can interact with keratin intermediate filaments. Filaggrin undergoes further processing in the upper stratum corneum to release free amino acids that assist in water retention.

“Fitzpatrick skin type” as used herein means a way to classify the skin by its reaction to exposure to sunlight as shown in Table 1 below.

TABLE 1

| Skin | | |
|---|---|---|
| Type | Typical Features | Tanning Ability |
| I | Pale white skin, blue/green | Always burns, does not tan |
| | eyes, blond/red hair | |
| II | Fair skin, blue eyes | Burns easily, tans poorly |
| III | Darker white skin | Tans after initial burn |
| IV | Light brown skin | Burns minimally, tans easily |
| V | Brown skin | Rarely burns, tans darkly easily |

Human-beta-defensin 1 (hBD1) is an antimicrobial peptide constitutively expressed by epithelial cells at mucosal surfaces and in the epidermis.

“Infant” as used herein refers to a human whose age ranges from birth to approximately twelve months of life.

“Inflammatory cytokine” is a type of signaling molecule that is secreted from immune cells and certain other cell types that promotes inflammation. Inflammatory cytokines are predominantly produced by T helper cells (Ths) and macrophages and involved in the upregulation of inflammatory reactions.

Interleukin-1 family (IL-1 family) is a group of 11 cytokines that plays a central role in the regulation of immune and inflammatory responses to infections or sterile insults.

IL-1RA (IL-1 receptor antagonist) is a natural antagonist of family members of IL-1.

Interleukin-36 gamma (IL-36gamma) is cytokine in the IL-1 family with pro-inflammatory effects.

“Metabolite” as used herein refers to the intermediate end product of metabolism. The term metabolite is usually restricted to small molecules. Metabolites have various functions, including fuel, structure, signaling, stimulatory and inhibitory effects on enzymes, catalytic activity of their own (usually as a cofactor to an enzyme), defense, and interactions with other organisms (e.g. pigments, odorants, and pheromones). A primary metabolite is directly involved in normal “growth”, development, and reproduction. A secondary metabolite is not directly involved in those processes, but usually has an important ecological function.

“Package” includes any suitable container for personal care regimens, ingredients and/or compositions.

“Personal care composition” as used herein, refers to compositions intended for topical application to the skin. The compositions used in accordance with the present disclosure include topically applied compositions, including leave-on formulations, and rinse-off formulations in which the product is applied topically to the skin and then is subsequently rinsed within minutes from the skin with water, or otherwise wiped off using a substrate with deposition of a portion of the composition. The personal care composition used in accordance with the present disclosure is typically dispensable from a package. Thus, in some embodiments, the dispensing may be by extruding. In some embodiments the package may be a single chamber package, or a multi chamber package, or a set of discrete packages. The personal care compositions used in accordance with the present disclosure can be in the form of liquid, semi-liquid, cream, lotion or gel intended for topical application to skin.

“P-value” is a measure of the probability that an observed difference could have occurred just by random chance. The lower the p-value, the greater the statistical significance of the observed difference. P-value can be used as an alternative to or in addition to pre-selected confidence levels for hypothesis testing. The standard level of significance used to justify a claim of a statistically significant effect is 0.05. The term statistically significant has become synonymous with p≤0.0.5. Bross IDJ (1971), “Critical Levels, Statistical Language and Scientific Inference,” in Godambe V P and Sprott (eds) Foundations of Statistical Inference. Toronto: Holt, Rinehart & Winston of Canada, Ltd.

A receiver operating characteristic (ROC) curve plots the true positive rate against the false positive rate at various thresholds. The ROC curve can inform performance of a binary classification algorithm.

“Rinse-off” ingredients or compositions by which is meant the ingredient or composition is applied topically to skin and then subsequently and immediately (i.e., within minutes) rinsed away with water, or otherwise wiped off using a substrate or other suitable removal means.

“Skin” is divided into three main structural layers, the outer epidermis, the inner dermis, and the subcutaneous tissue.

“Stratum corneum” as used herein, refers to the outermost layer of the epithelia, or the epidermis, and is the skin structure that provides a chemical and physical barrier between the body of an animal and the environment. The stratum corneum is a densely packed structure comprising an intracellular fibrous matrix that is hydrophilic and able to trap and retain water. The intercellular space is filled with lipids formed and secreted by keratinocytes and which provide a diffusion pathway to channel substances with low solubility in water.

“Subject” as used herein refers to a human for whom a regimen, ingredient and/or composition is tested or on whom a regimen, ingredient and/or composition is used in accordance with the methods described herein.

“Substantially free of” as used herein, unless otherwise specified, means that the personal care regimen, ingredient and/or composition comprises less than about 2%, less than about 1%, less than about 0.5%, or even less than about 0.1% of the stated ingredient. The term “free of”, as used herein, means that the personal care regimen, ingredient and/or composition comprises 0% of the stated ingredient. However, these ingredients may incidentally form as a by-product or a reaction product of the other components of the personal care regimen, ingredient and/or composition.

“Test ingredients and/or compositions” as used herein include and encompass purified or substantially pure ingredients and/or compositions, as well as formulations comprising one or multiple ingredients and/or compositions. Thus, non-limiting examples of test ingredients and/or compositions include water, a pharmaceutical or cosmeceutical, a product, a mixture of compounds or products, and other examples and combinations and dilutions thereof.

“Test surfaces” as used herein means a region of epithelia tissue which has been contacted with and/or by a product, such as a consumer product and/or a test regimen, ingredient and/or composition, whereby the contact of the product and/or the regimen, ingredient and/or composition on the epithelia tissue has resulted in some change, such as but not limited to, physiological, biochemical, visible, and/or tactile changes, in and/or on the epithelia tissue that may be positive or negative. In some examples, positive effects caused by regimen, ingredient and/or composition may include but are not limited to, reduction in one or more of erythema, trans-epidermal water loss (TEWL), discoloration of the skin, rash, dermatitis, inflammation, eczema, dandruff, edema and the like. The location of the affected surface will depend upon the regimen, ingredient and/or composition used or the location of some physiological, biochemical, visible, and/or tactile change in and/or on the epithelia tissue.

“Topical application”, “topically”, and “topical”, as used herein, mean to apply the regimen, ingredient and/or composition used in accordance with the present disclosure onto the surface of the skin.

“Treating” or “treatment” or “treat” as used herein includes regulating and/or immediately improving skin appearance and/or feel.

“Young child/children” as used herein refers to a human/humans whose age ranges from approximately twelve months of life to approximately 3 years, or approximately 5 years, or approximately 7 years of life.

“Z-score” is the number of standard deviations by which the value of a raw score is above or below the mean value of what is being observed or measured. It (a) allows one to calculate the probability of a score occurring within normal distribution; and (b) enables one to compare two scores that are from different normal distributions.

### SUMMARY OF THE INVENTION

The invention relates to factors that can be used to predict the propensity of an individual to develop atopic dermatitis. In accordance with the invention, the method comprises:
a) observing expression of a biomarker selected from hBD1, IL1RA, IL36g and S100A8/9 or combinations thereof on the skin surface;
b) comparing said expression to a determined standard, wherein said determined standard is ascertained by measuring a level of said biomarker in a subject or pool of subjects who have demonstrated an absence of atopic dermatitis; and
c) determining the propensity of an individual to develop atopic dermatitis, wherein an increase in the expression as compared to said determined standard indicates a propensity of said individual to develop atopic dermatitis.

The individual may be selected from the group consisting of infants and young children.

The skin may be selected from face skin and/or body skin.

Also in accordance with the invention, a method for evaluating the efficacy of a skin treatment regimen, ingredient and/or composition to treat atopic dermatitis, comprises:
(a) measuring the level of hBD1, IL1RA, IL36g and S100A8/9 on an area of skin prior to application of the skin treatment regimen, ingredient and/or composition;
(b) applying the skin treatment regimen, ingredient and/or composition to the area of skin for a period of time;
(c) measuring the level of hBD1, IL1RA, IL36g and S100A8/9 on the skin area after the skin treatment regimen, ingredient and/or composition application on the area of skin;
wherein the skin treatment regimen, ingredient and/or composition is beneficial to the skin if the level of the hBD1, IL1RA, IL36g and S100A8/9 is less than or equal to the no treatment control.

These and any other methods, skin treatment regimens, ingredients and/or compositions will be described in more detail below.

The present inventors have identified four inflammatory cytokines (hBD1, IL1RA (interleukin 1 receptor antagonist), IL36g, S100A8/9) on the body that can be used to distinguish high-risk subjects capable of developing AD within 12 months vs. low risk that will most likely not develop AD. A combined Z score for the cytokines was generated for each sample by summing up the Z scores either from 14 cytokines or the 4 cytokines mentioned above. Both the combined Z score with all cytokines or the combined Z score with the 4 cytokines was significantly different between converters vs non-converters at baseline (p<0.05).

### Converter Data: Microbiome

In addition to the dysregulated inflammation observed in converters, we also identified differentially expressed skin microbes that could distinguish converters and non-converters at baseline. We identified a total of 19 downregulated species on the body, and 6 upregulated and 26 downregulated species on the face (FDR<0.01). See Table 2 below. Downregulated species included skin commensals such as S. epidermidis and S. lugdunensis, both of which are known to reduce S. aureus colonization.

TABLE 2

| Body significant species | | | Face significant species | | |
|---|---|---|---|---|---|
| Species | log2FoldChange | Padj | Species | log2FoldChange | Padj |
| Staphylococcus lugdunensis | −17.85786741 | 9.88E−38 | Pauljensenia odontolytica_A | −19.66768423 | 4.00E−49 |
| Gordonia jacobaea | −18.76444255 | 1.14E−19 | Fusobacterium periodonticum | −16.37055918 | 1.93E−32 |
| Paraprevotella clara | −17.63432891 | 1.26E−16 | Neisseria zoodegmatis | −18.2183778 | 2.01E−18 |
| Rhodococcus erythropolis_D | −20.62813535 | 4.55E−16 | Stenotrophomonas rhizophila_A | −18.48506175 | 1.99E−15 |
| Pseudomonas_A sp004010935 | −18.07711433 | 5.90E−15 | Clostridium_P perfringens | −17.37666324 | 6.61E−15 |
| Pseudoduganella armeniaca | −17.32725671 | 5.01E−08 | Pantoea brenneri | 10.4315945 | 1.10E−14 |
| Carnobacterium gallinarum | −33.8083732 | 2.55E−06 | Pseudomonas_A sp003205815 | −16.69205065 | 2.33E−10 |
| Rothia sp004136585 | −31.92556288 | 5.10E−06 | Stenotrophomonas sp003086895 | −15.92231724 | 1.19E−09 |
| Chryseobacterium oranimense | −31.46344629 | 1.75E−05 | Stenotrophomonas maltophilia | −14.19077404 | 1.19E−09 |
| Corynebacterium massiliense | −30.28804856 | 4.26E−05 | Pantoea dispersa | 8.598885644 | 1.19E−09 |
| Lactococcus sp003627095 | −28.77704059 | 4.26E−05 | Pararheinheimera mesophila | −8.58189816 | 1.34E−09 |
| Gordonia paraffinivorans | −28.34515399 | 4.52E−05 | Pediococcus stilesii | −24.60672179 | 1.95E−08 |
| Nissabacter sp002858675 | −29.67046667 | 5.78E−05 | Tatumella citrea | 35.80099953 | 2.01E−07 |
| Weissella paramesenteroides | −28.24046793 | 0.00016362 | Staphylococcus aureus | −6.598394726 | 1.09E−06 |
| Lacticaseibacillus casei | −16.02750241 | 0.00016362 | Staphylococcus xylosus_A | −14.36606243 | 5.65E−06 |
| Rhodobacter aestuarii | −26.17977961 | 0.0007671 | Pseudomonas_E straminea | 31.91750121 | 6.61E−06 |
| Staphylococcus xylosus_A | −13.65591505 | 0.00157126 | Staphylococcus epidermidis | −3.337705296 | 7.76E−06 |
| Ancylobacter rudongensis | −24.94006676 | 0.00170448 | Vagococcus fluvialis_A | −31.48608644 | 8.91E−06 |
| Paracoccus thiocyanatus | −24.30777533 | 0.00253457 | Novosphingobium sp900113255 | −17.68599586 | 1.34E−05 |
| | | | Vagococcus fluvialis | −28.07614014 | 0.000144235 |
| | | | Gluconobacter japonicus | −14.36767292 | 0.000217659 |
| | | | Cutibacterium namnetense | −2.719113676 | 0.000217659 |
| | | | Pantoea ananatis | 6.83929002 | 0.000217659 |
| | | | Corynebacterium kroppenstedtii | −4.155698755 | 0.000225697 |
| | | | Moraxella catarrhalis | −6.52450665 | 0.000382631 |
| | | | Acidipropionibacterium jensenii | −3.649438573 | 0.000749952 |
| | | | Streptococcus mitis_BJ | −3.120209278 | 0.00228289 |
| | | | Stenotrophomonas | −19.1423783 | 0.002339563 |
| | | | maltophilia_Q | | |
| | | | Streptococcus sp000235485 | −3.159935613 | 0.002620217 |
| | | | Actinomyces massiliensis | −3.143949101 | 0.004700488 |
| | | | Pantoea sp002920175 | 6.105051779 | 0.007104492 |
| | | | Actinomyces oris_A | −3.521647046 | 0.009460515 |

Statistical difference was observed between converters and non-converters in the total amount of amino acids as measured by Raman spectroscopy.

Non-lesional skin of AD patients is characterized by weaker barrier and less amino acid content compared to healthy subjects

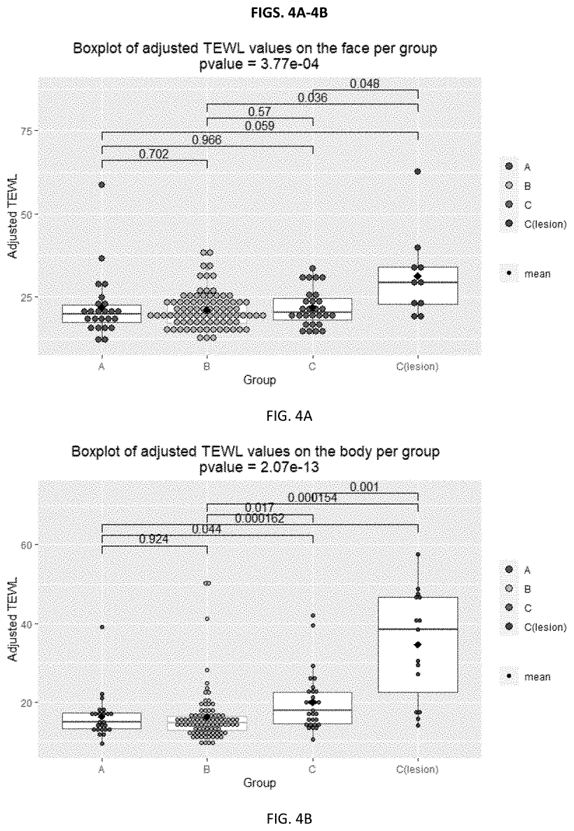

Non-lesional skin on the face of AD subjects had higher TEWL rates compared to corresponding skin sites of either the low- or high-risk non-AD groups. Although TEWL measurements were not sensitive enough to show that this is also the case on the body, the water transport resistance profiles calculated from the water concentration profiles within the SC confirmed that skin barrier is weaker on AD subjects for the arm site as well (FIGS. 4A and 4B). Importantly, the area under the curve of the water transport resistance profile was statistically different between the low- and the high-risk groups, with the latter showing a weaker barrier (FIG. 4B).

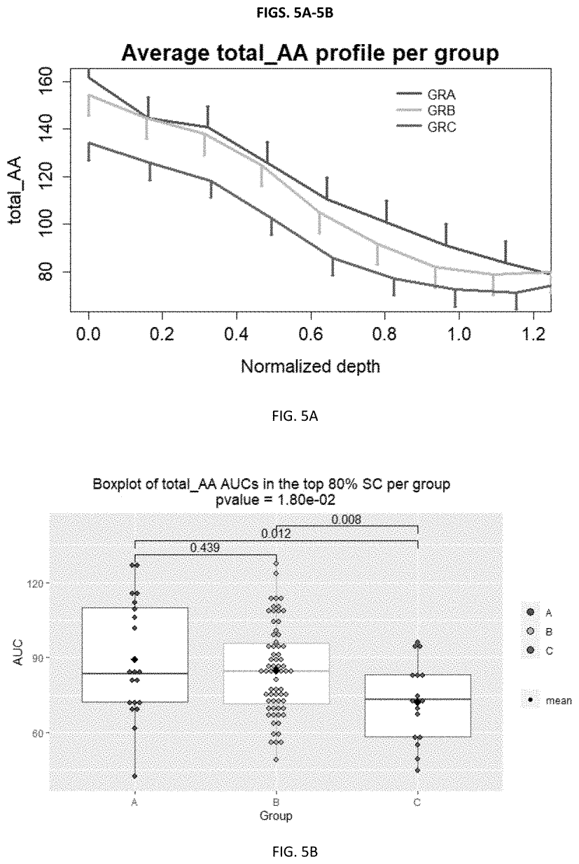

Raman measurements also revealed less total amino acid content in AD patients (non-lesional skin) compared to the two non-AD groups (FIGS. 5A and 5B), an observation that indicates less efficient proteolysis of filaggrin-like molecules in the SC of AD patients.

### DISCUSSION

### Study Design

A single-center exploratory study was conducted to compare skin biomarkers in 3 groups of children aged between 3 and 48 months, based on their status of atopic dermatitis and their family history of atopic disease.

All subjects were healthy, Caucasian, with a Fitzpatrick skin type between Ito III and residents of the Hamburg, Germany area.

Prior to the study the caregivers were instructed not to apply any leave on cosmetics (e.g. creams, lotions, oily cleansing products) on their child in the test area within the last 3 days prior to the start of the study or apply any detergents (e.g. soaps, shampoos, bath and shower products) on their child in the test area at the day of the start of the study. They were instructed to avoid contact of the test area with water (e.g. no bathing, no swimming, no showering) within the last 2 hours prior to the instrumental measurements. The caregivers filled in questionnaires to provided information for demographic data: age, gender, ethnicity, Fitzpatrick skin types and mode of birth (vaginal or C-section).

Subjects were divided into the following groups:

Group A—29 enrolled subjects: Healthy/Low Risk—Without atopic background for either parent or siblings (without history of atopic dermatitis, hay fever or allergic asthma).

Group B—101 enrolled subjects: Healthy/High Risk—With atopic background for either parent or siblings (with history of atopic dermatitis, hay fever or allergic asthma).

Group C—30 enrolled subjects: Active atopic dermatitis with local SCORAD (1) of 4-8 as assessed by a pediatrician.

It was aimed for obtaining about 30 valid data sets of each parameter for subgroups A and C and about 100 valid data sets of each parameter for subgroup B, but this could not be guaranteed due to the mood/behavior of the very young children. Subjects who dropped out after enrollment into the study were not replaced.

Children with serious illnesses or systemic medication past or present, any skin related conditions, including atopic dermatitis (beside for group C), seborrheic dermatitis, psoriasis or skin infections were excluded from the study.

### Measurements

All measurements took place in an air-conditioned room at a temperature of 21±1° C. and at 50±5% relative humidity. Before measurements, the subjects stayed in the climatized room for at least 30 min.

The measurement of stratum corneum hydration was performed by the electrical capacitance method (CORNEOMETER CM 825, Courage & Khazaka, Cologne, Germany) given in arbitrary units (AU)). Five measurements were performed at each test area.

Skin barrier was assessed by measuring the transepidermal water loss (TEWL) with a closed chamber system (AQUAFLUX AF200, Biox Systems Ltd., London, UK). TEWL was given in g/m²h and a single measurement was performed at each test area.

Magnified photos of the test area were acquired using a high-resolution digital camera (Canon EOS 5D Mark III; 50 mm Macrolens/28 mm Macrolens) in combination with a dermatoscope (DERMlite TM Foto, 3Gen Inc., San Juan Capistrano, Calif., USA) with ring-light illumination and distance holder. The captured test area was 1.5 to 2 cm/2 to 2.5 cm (28 mm lens) in diameter. For color consistency, white balancing and color calibration was performed using the X-Rite Color Checker. A single image per test area was acquired.

Raman spectra at consecutive depths in the skin were acquired using an in vivo confocal Raman microspectrometer (gent-SCA Skin Analyzer, River D, Rotterdam, The Netherlands). Concentration profiles of water (wavenumber range 2600-3800 cm⁻¹) and NMF components (wavenumber range 400-1800 cm⁻¹) were calculated from the Raman spectra using the manufacturer's software (SkinTools). For water profiles spectra were acquired from the skin surface up to a depth of approx. 48 μm in the skin, in steps of 2.0 μm with an integration time of 1 sec and a 25 μm diameter pinhole. Approximately 6 profiles were measured per test area. For NMF profiles spectra were acquired from the skin surface up to a depth of approx. 48 μm in the skin, in steps of 2.5 μm with an integration time of 5 sec and a 100 μm diameter pinhole. Approximately 6 profiles were measured per test area.

Defined skin areas (4 cm×4 cm) were swabbed with a COPAN 502cs01 flocked swab soaked in molecular grade DNA free water (PCRH2O-RO—Water, PCR Grade, Sigma-Aldrich) on the cheek and elbow pit/lesion. For risk group C, assessments were additionally performed on healthy skin beside the lesional skin. The swab was rubbed back and forth approximately 50 times applying firm pressure under slow rotation. The head of the swab was then aseptically cut from the handle and stored in a sterile Eppendorf tube (without buffer). Samples were stored on dry ice until further processing and at −80 ° C. until shipment. To minimize sample cross-contamination, a fresh pair of gloves was worn by the person sampling each individual. Microbiome sequencing using primers targeting the 16S regions was conducted.

Inflammatory marker analysis was performed by swabbing the skin areas of interest using specialized swabs soaked in buffer (FibroTx, Tallinn, Estonia). Samples were stored on dry ice until further processing and at −80 ° C. until shipment to FibroTx for marker analysis using a spot enzyme-linked immunosorbent assay. The samples were analyzed for the presence of CCL-17, CCL-2, CCL-22, CL-27, CXCL-1/2, hBD-1, IL-13, IL-18, IL-la, IL-1b, IL-1RA, IL-36g, IL-8, 5100A8/9, and VEGF-A.

Measurements by Raman Confocal Spectroscopy were performed on the volar forearm (left or right side randomized). For risk group C assessments were performed preferably on the atopic dermatitis lesion, if possible. Measurements for TEWL, skin surface hydration, imaging, and microbiome and inflammatory marker sampling were performed on the arm and on the center of the cheek (left or right side randomized). FibroTx Swabs were preferably performed on the other side or next to the area where the instrumental measurements and the Swabs for microbiome analysis took place. For risk group C, assessments were additionally performed on healthy skin beside the lesional skin in the elbow pit. Local SCORAD for risk group C was assessed on a suitable body region, preferably in the elbow pit.

### Statistical Analyses

All statistical analyses were performed on R using the packages: DescTools, rstatix, and ggplot2.

Outliers identified as falling in the interval [q₂₅−1.5*IQR; q₇₅+1.5*IQR] were removed, where q₂₅ quantile at 25% per group, q₇₅ quantile at 75% per group, and IQR=q₇₅−q₂₅ the Inter-Quantile Range per group.

To account for differences in gender, age, and birth method between groups, a linear statistical model of the calculated AUCs was built and adjust them with respect to these 3 variables according to:

Xₗₘ_{_{i,j}}˜a₀a₁*genderᵢ+a₂*ageᵢ+a₃*birth methodᵢ (1)

where Xₗₘ_{_{i,j}} is variable X (AUC, Total Amino acids, TEWL or Hydration measures) as calculated by the linear model for subject i at measuring site j (body or face), genderᵢ is subject i's gender, ageᵢ is subject i's age, birth methodᵢ is subject i's birth mode of delivery, aₖ linear model's parameters, and

X_{adjusted}_{_{i,j}}˜Xₗₘ_{_{i,j}}−a₀−aₐ*genderᵢ−a₂*ageᵢ−a₃*birth methodᵢ (2)

where X_{adjusted}_{_{i,j}} is variable X adjusted with respect to gender, age, and birth method for subject i at measuring site j (body or face). Group comparisons for the adjusted variables were tested using ANOVA and pairwise Bonferroni tests.

Raman spectra per risk group were plotted with respect to location in the stratum corneum (surface location is set to 0). Water profiles that did not reach a plateau in the viable epidermis were removed. Water resistance profiles were computed using water concentration profiles and corresponding TEWL values, as described in (van Logtestijn M D, 2015). To evaluate the difference between risk groups, the Area Under the Curve (AUC) was computed for each water resistance profile over the stratum corneum (SC) thickness (normalized depth between 0 and 1).

For the spectra collected in the fingerprint region, outliers of spectral stacks were determined using the Keratin profiles and removed from subsequent analysis. The calculated concentration profiles were normalized by SC thickness for each subject. Total Amino Acids (AAs) profiles were obtained from the sum of the amino acid component profiles (as calculated by SkinTools, described above). Average total AAs profiles per group were computed by averaging data points at a certain depth for all subjects in a risk group. To evaluate the difference between the 3 risk groups, the AUC for each total AAs profile was computed in the top 80% of the SC, i.e. normalized depth between 0 and 0.8

Differential Expression of inflammatory cytokines was performed separately for the face and body using a linear model that incorporated Age, Sex, and Birth mode as covariates. Combined Z score was generated by summing up the individual Z scores of all measured cytokines. Correlation between different variables was performed using Spearman's rank correlation.

Machine learning models were generated by training models to classify AD lesional tissue vs. healthy low risk samples using all 15 cytokines. Four machine learning algorithms (Elastic Net, Random Forest, Support Vector Machine, XGBoost) were trained with 5-fold cross-validation repeated 5 times. The model was tested by classifying healthy high-risk converters vs. non-converters using the same 15 cytokines. Separate models were generated for the face and the body.

16S microbiome data was first filtered using SHI7 with lenient settings (https://journals.asm.org/doi/10.1128/mSystems.00202-17) and reads were stitched with FLASH (https://www.ncbi.nlm.nih.gov/pmc/articles/PMC3198573/). The merged reads were then aligned by BURST aligner (v1.00; https://www.biorxiv.org/content/10.1101/2020.09.08.287128v1) with 93.5% per read specificity cutoff and capitalist setting to GTDB95 prokaryote SSU DB (https://www.nature.com/articles/nbt.4229) to assign taxonomy.

After the reads were aligned, samples with <1k reads were filtered out, and taxa expressed in <1% of total samples were removed. Differential expression was performed using DESEQ2 with age, sex, and birth mode as covariates.

It will be understood that, while various aspects of the present disclosure have been illustrated and described by way of example, the invention claimed herein is not limited thereto, but may be otherwise variously embodied according to the scope of the claims presented in this and/or any derivative patent application.

1. A method of predicting a propensity of an individual to develop atopic dermatitis, comprising:
a) observing the expression of a biomarker selected from hBD1, IL1RA, IL36g and 5100A8/9 or a combination thereof on a skin area;
b) comparing said expression to determined standard, wherein said determined standard is ascertained by measuring a level of said biomarker in a subject or pool of subjects who have demonstrated an absence of atopic dermatitis; and
c) determining the propensity of an individual to develop atopic dermatitis,
wherein an increase in the expression as compared to said determined standard indicates propensity of said individual to develop atopic dermatitis.

2. The method of claim 1, wherein the individual is selected from the group consisting of infants and young children.

3. The method of claim 1, wherein the skin is selected from face skin and body skin.

4. A method for evaluating the efficacy of a skin treatment regimen, ingredient and/or composition to treat atopic dermatitis, comprising:
(a) measuring the level of hBD1, IL1RA, IL36g and 5100A8/9 on an area of skin prior to application of the skin treatment regimen, ingredient and/or composition;
(b) applying the skin treatment regimen, ingredient and/or composition to the area of skin for a period of time;
(c) measuring the level of hBD1, IL1RA, IL36g and 5100A8/9 after the skin treatment regimen, ingredient and/or composition application on the area of skin;
wherein the skin treatment regimen, ingredient and/or composition is beneficial to the skin if the level of the hBD1, IL1RA, IL36g and 5100A8/9 is less than or equal to the no treatment control.

5. A method of predicting a propensity of an individual to develop atopic dermatitis, comprising:
a) observing the expression of at least one skin microbe species selected from those in Table 2 on a skin area;
b) comparing said expression to a determined standard, wherein said determined standard is ascertained by measuring a level of said at least one skin microbe in a subject or pool of subjects who have demonstrated an absence of atopic dermatitis; and
c) determining the propensity of an individual to develop atopic dermatitis,
wherein an increase in the expression as compared to said determined standard indicates propensity of said individual to develop atopic dermatitis.

6. A method of predicting a propensity of an individual to develop atopic dermatitis, comprising:
a) observing total amino acid content on a skin area;
b) comparing said total amino acid content to a determined standard, wherein said determined standard is ascertained by measuring a level of said total amino acid content in a subject or pool of subjects who have demonstrated an absence of atopic dermatitis; and
c) determining the propensity of an individual to develop atopic dermatitis,
wherein a decrease in total amino acid content as compared to said determined standard indicates propensity of said individual to develop atopic dermatitis.

7. A method of predicting a propensity of an individual to develop atopic dermatitis, comprising:
a) observing two or more parameters selected from the group consisting of (i) the expression of a biomarker selected from hBD1, IL1RA, IL36g and S100A8/9 or a combination thereof on a skin area; (ii) the expression of at least one skin microbe species selected from those in Table 2 on a skin area; and (iii) the total amino acid content on a skin area;
b) comparing said two or more parameters to a determined standard for each of said two or more parameters, wherein each of said determined standards is ascertained by measuring a level of said respective parameter in a subject or pool of subjects who have demonstrated an absence of atopic dermatitis; and
c) determining the propensity of an individual to develop atopic dermatitis,
wherein a change in said parameter as compared to said determined standard indicates propensity of said individual to develop atopic dermatitis.
